(19) United States
(12) Patent Application Publication
(10) Pub. No.: US 2026/0224291 A1
(43) Pub. Date: Aug. 6, 2026

(54) SURGICAL ASSISTANCE SYSTEM, AND DATA VISUALISATION METHOD FOR A SURGICAL PROCEDURE

(71) Applicant: B. Braun New Ventures GmbH, Freiburg im Breisgau (DE)

(72) Inventor: Jean Stawiaski, Kirchzarten (DE)

(21) Appl. No.: 19/151,078

(22) PCT Filed: Jan. 25, 2024
(86) PCT No.: PCT/EP2024/051812

§ 371 Date: Jul. 25, 2025

(30) Foreign Application Priority Data
Jan. 26, 2023 (DE) 10 2023 101 953.8

(51) Int. Cl. A61B 34/20; A61B 1/00; A61B 34/00; A61B 34/30; B25J 9/16

(52) U.S. Cl. CPC A61B 34/20; A61B 1/00045; A61B 1/00149; A61B 34/25; A61B 34/30; B25J 9/1679; A61B 2034/301

(57) ABSTRACT

A surgical assistance system includes a navigated surgical robot for use in a surgical procedure on a patient, a robot base as a local connection point of the robot, and a movable robot arm connected to the robot base. The robot arm has at least one robot arm segment. A visualization system is connected to the robot arm and adapted to generate and provide at least one real-time intracorporeal image. A navigation system is adapted to determine, with spatial reference to a patient, at least one position of the visualization system, and thus a position of the at least one real-time intracorporeal image. A control unit is adapted to assign and store a position in a coordinate system of the patient to the real-time intracorporeal image. The system can be used in conjunction with a method for data visualization and with a computer-readable storage medium.

### CROSS-REFERENCE TO RELATED APPLICATIONS

This application is the United States national stage entry of International Application No. PCT/EP2024/051812, filed on Jan. 25, 2024, and claims priority to German Application No. 10 2023 101 953.8, filed on Jan. 26, 2023. The contents of International Application No. PCT/EP2024/051812 and German Application No. 10 2023 101 953.8 are incorporated by reference herein in their entireties.

### FIELD

The present disclosure relates to a surgical assistance system, in particular a neurosurgical assistance system, including a navigated surgical robot for use in a surgical procedure on a patient. The robot has a robot base as a local connection point of the robot and, in a sense, as a local fixed coordinate system. A movable robot arm that includes at least one robot arm segment is connected to the robot base. In addition, the assistance system has a visualization system with one or more cameras which is connected to the robot arm, in particular at a terminal side of the robot arm, and in particular is mounted there. The visualization system is adapted to create at least one real-time, corporeal, preferably intracorporeal image of the patient and to provide it, preferably in digital form. Using a navigation system of the assistance system, at least the location, i.e., a position and orientation, of the visualization system and thus the location of the at least one real-time, corporeal image can be determined with spatial reference to the patient. As a rule, a manually guided surgical instrument or a surgical instrument guided by the navigated surgical robot also is provided. If this is the case, its position in relation to the patient preferably can also be tracked by the navigation system. Moreover, a control unit is provided. This is at least adapted to assign a position in a coordinate system of the patient to the at least one real-time, intracorporeal image and to store it. It is preferably also adapted to control the aforementioned subsystems of the assistance system, i.e., to control at least the robot arm, the visualization system, and the navigation system, and to control their interaction.

### BACKGROUND

In the field of medicine and medical technology, automation with the integration of digitally controllable technical devices is becoming more and more important. Robots are increasingly being used in surgical procedures, where they are particularly useful in supporting precise, minimally invasive procedures. In this context, the robot is not only intended to be a stand-alone robot that performs the operation alone, but is increasingly being used as a collaborative robot (cobot), i.e., an assisting or supporting robot, directly in the surgical field, interacting with medical personnel, in particular the surgeon.

As a rule, various visualization and measurement systems are used for such robot-assisted surgical procedures, especially neurosurgical procedures on the brain, for visual, physiological, and functional assessment of the surgical site and as a decision-making aid. The classic data collected before and during the procedure include for example, preoperative MRI and CT images of the patient, position data of the surgical instruments provided by the navigation system, images from a multimodal digital microscope, data from the robot for precise positioning of the surgical instruments and the visualization system, electrophysiological monitoring data, histological samples, intraoperative ultrasound images, and the like.

Existing surgical assistance systems, especially their navigation systems, can only summarize a limited amount of some of the data listed above. Preoperative patient scans are used as a common reference. The most advanced navigation systems can be used to localize and navigate the digital microscope so that the position of the surgical instruments and the real-time images (live images) captured by the microscope during the operation can be merged and displayed together.

On the other hand, current robot visualization systems enable the position of a robot arm to be linked to the images of a microscope that is permanently connected to the robot's end effector. These systems thus make it possible to precisely reproduce the recording conditions of microscopic images. This important feature of robot visualization systems is often referred to as a “waypoint.” The waypoint is defined and stored as at least a specific posture of the robot arm and its segments relative to each other, for example in the form of a set of joint angles. Stored waypoints can be recalled by the user, allowing the robot arm to be moved back exactly to the stored posture and the visualization condition of the relevant camera that is represented by the waypoint to be reproduced exactly. In addition, it is possible to rotate or translate the visualization system around a stored point on the patient, known as the “locked target.” However, the existing waypoint functionality for the visualization system and the surgical navigation system currently are not integrated, so that navigation of the current image and the instrument is not consistent. As a result, the existing waypoint functionality is unable to compensate for unwanted movement or position changes of the patient.

This makes things difficult for the surgeon, as the data from the visualization system and the navigation system have to be tracked on different systems, each with separate displays. This is further complicated by the fact that additional external data relevant to the operation, which cannot be visualized in real time, for example, because it was recorded preoperatively or during the operation and must first be processed externally, or which is located outside the area of the procedure, must be tracked on additional systems. Typical examples of this are histological data (location-dependent or location-independent) or electrophysiological data (quality of a local (electrochemical) signal transmission). These are neither integrated into the navigation system nor into a robot positioning system, which makes their availability and interpretation considerably more difficult. Furthermore, this data cannot be easily presented to a surgeon, as several different systems and screens must be used, as mentioned above. This is a major disadvantage of existing surgical assistance systems. Moreover, it is known that data relevant to surgery can be organized and stored along a timeline. However, the surgeon can only examine this data in a post-operative environment, based on the time at which it was recorded.

### SUMMARY

In contrast, it is the object of the present disclosure to avoid, or at least reduce the disadvantages of the prior art, and in particular provide a surgical assistance system, a (computer-implemented) method for data visualization including a surgical assistance system, and a computer-readable storage medium that allows a user to interpret operation-relevant data during surgery in a particularly simple, intuitive, and safe manner. One task in particular is to process, combine, and display different modalities of medical information in such a way that they can be clearly displayed on a single display as needed, the relevant combined information for a specific tissue area of the patient also being displayed for that exact tissue area.

The tasks are solved by a surgical assistance system, by a (computer-implemented) method for data visualization, and by a computer-readable storage medium, according to the present disclosure.

A basic idea of the present disclosure is to create a surgical assistance system that is adapted to enable the spatial arrangement or assignment of operation-relevant data of any data modality and to jointly display this data spatially. In the sense of the disclosure, the data modality describes a respective method for data collection. The data modality can be, for example, imaging or functional, on the patient or on the assistance system, and/or real-time or preoperative or postoperative. The spatial arrangement of this data is preferably made possible with a spatial reference to the patient, in particular directly to a patient's anatomy, preferably with reference to a patient coordinate system. This joint arrangement and display in accordance with the disclosure allows for easier, more intuitive, and safer interpretation of all operation-relevant data by a user during surgery, regardless of its data modality.

In other words, a surgical assistance system is provided that is adapted to merge or aggregate operation-relevant data of different data modalities that are recorded during, before, and/or after an operation. A surgical navigation system of the assistance system is provided to spatially locate and visualize the heterogeneous data based on their different data modalities. The data are linked to each other by their relative position to the patient, in particular to the patient's coordinate system.

Specifically, a surgical assistance system, in particular a neurosurgical assistance system, comprises a navigated surgical robot for use in a surgical procedure on a patient. The robot has a robot base as a local connection point of the robot and a movable robot arm connected to the robot base with at least one robot arm segment. A visualization system (or visualization unit) is connected, in particular mounted, on the robot arm, in particular on a terminal side of the robot arm. This is adapted to produce at least one real-time, intracorporeal image of the patient and to subsequently make it available in digital or computer-readable form. The assistance system moreover has a navigation system that is adapted to determine, with spatial reference to the patient, at least a location, i.e. a position and orientation, of the visualization system and thus (for example, via a transformation from the visualization system to the image, for example by means of depth information) the location of the at least one real-time, intracorporeal image. For this purpose, the navigation system may in particular comprise a 3D camera and rigid bodies (with markers) tracked by the camera, at least one of which is firmly coupled to the visualization system. The location of the visualization system can be determined in particular by a triangulation of 3D images of this rigid body, preferably performed in a control unit of the assistance system. Alternatively or in addition, the navigation system may comprise a detection unit for detecting joint angles of the robot arm (i.e., the configuration of the robot arm segments), whereby the location of the visualization system can in turn be determined via the control unit if the kinematics of the robot arm are otherwise known. Preferably, the assistance system also has at least one surgical instrument that is guided manually or by the navigated surgical robot, the location of which, in particular a location of an end effector of the instrument, is tracked by the navigation system in relation to the patient. A control unit of the assistance system is specially adapted to assign at least one position, in particular a location in a coordinate system of the patient, to the at least one real-time intracorporeal image and to store it, preferably in a data information system of the control unit. The at least one real-time, intracorporeal image has a first data modality resulting from the acquisition method. According to the present disclosure, the control unit is adapted to import further operation-relevant data, preferably of the patient and/or the assistance system, which are acquired with a second data modality different from that of the visualization system, into the data information system via a data interface. The data of the second data modality can be recorded preoperatively and/or in real time (and/or even postoperatively, for example for controlling the operation). Thus, the at least one real-time, intracorporeal image of the patient and other operation-relevant data of any data modality are stored in the data information system. Moreover, the control unit is adapted to assign a position to the imported data in the patient's coordinate system and to store it in the data information system. Figuratively speaking, the data with the second data modality are spatially attached to the (virtual) patient. This means that all data relevant to the operation are assigned a spatial position in the patient's (central) coordinate system. The data information system thus forms a basis for the joint, spatial representation of both the image(s) and the other data, regardless of their data modality. Even data that is not spatially located on the patient, such as operating data from the robot, or data of the patient that is not real-time, such as preoperative scans or functional measurement data, is thus assigned at least a spatial position on the patient. Finally, the control unit is adapted to generate a view of the at least one real-time intracorporeal image together with the imported data and to output this view via a display device of the assistance system, in particular a surgery monitor. Preferably, the control unit is adapted to generate this view of the at least one real-time intracorporeal image only with those of the imported data that are spatially located within the at least one real-time intracorporeal image due to their position.

Thus, in particular the central coordinate system creates a central reference point into which all medical data from different modalities can be integrated, for example to process different information centrally and achieve synergistic views (such as overlays, augmented reality, and the like), links, or correlations, and to output the patient's important medical information centrally for the surgeon.

This provides a solution for bringing data relevant to the operation that, due to its data modality, are not (intracorporeal) recordings or image data of the patient into the same field of view as live images, recordings, or other image data. It is displayed in the best possible reference system, namely the patient's body. Thus, the output or display of operation-relevant data that differs in its data modality can be shown on a single, joint display device. The effect for the surgeon is that he/she no longer has to look at multiple displays, but merely on a single display to see all operation-relevant data. Both the output of the data on a single display device and the spatial representation of all operation-relevant data on the patient enable simpler, more intuitive, and safer interpretation of the operation-relevant data during surgery. This leads to safer surgery and a reduction in the duration of the procedure.

Thus, a surgical assistance system is created that avoids or at least reduces the disadvantages of the prior art mentioned above.

In the present disclosure, the term “end effector” refers to a device, instrument, or similar medical device that can be used to perform a procedure on a patient. In particular, the following can be regarded as end effectors: an instrument, a medical device such as an endoscope or a suction tube, an optical device with a visualization axis, a pointer with a distal tip for surgical navigation, and others.

The term “robot arm segment” here refers in particular to a robot part of the robot arm mounted between bearings or joints or, in the case of the robot arm segment on the terminal side, in particular a robot part connected in series between the end effector and the preceding robot arm segment (in the case of only one robot arm segment correspondingly the robot base).

The term “position” refers to the geometric position in three-dimensional space, which is specified in particular by means of coordinates of a Cartesian coordinate system. In particular, the position can be specified by the three coordinates X, Y, and Z.

The term “orientation” in turn indicates an alignment (for example, at the position) in space. It can also be said that the orientation specifies an alignment with an indication of direction and rotation, respectively, in three-dimensional space. In particular, the orientation can be specified by way of three angles.

The term “location” encompasses both a position and an orientation. In particular, the location can be specified by means of six coordinates, three position coordinates X, Y, and Z, and three angle coordinates for the orientation.

Advantageous embodiments are explained especially below.

According to a preferred embodiment, the visualization system may comprise an overview camera with a field of view that is provided and configured to capture the intervention area and its surroundings, and/or a microscope camera (in particular with magnification and a zoom function) with a field of view that is provided and adapted to capture a surface of the intervention or of a tissue, and/or an endoscope camera with a field of view that is provided and designed to capture intracorporeal images, in particular below a surface of the intervention area.

According to a preferred embodiment, the assistance system may be adapted to register the patient via the navigation system so that the navigation system is set up to navigate the instruments and/or the visualization system.

According to a preferred embodiment, any of the aforementioned components or cameras of the visualization system may be calibrated with reference to the patient's coordinate system.

Respective intrinsic parameters (of the visualization units), such as in particular a focal length, a zoom, distortion coefficients and the like, may preferably be stored together with the respective images in the data information system.

The data recorded using the second data modality are preferably image data or scans of the patient taken prior to surgery, in particular a CT scan and/or an MRI scan and/or a histological image and/or a 3D image. Alternatively or in addition, measurement data of the patient, in particular electrophysiological data, and/or operating data of the surgical robot, such as in particular a set of joint angles of the robot arm or information about the currently used end effector, its nominal data or operating parameters such as torque, speed or the like, may be recorded.

In order to import electrophysiological measurement data, the assistance system according to a preferred embodiment, may have a serial interface. An interface in accordance with the Digital Imaging and Communications in Medicine (DICOM) standard may preferably be provided for importing histological measurement data.

In order not to overload the view with data, the control unit is adapted in a further development to continuously determine the coordinates of the field of view of the at least one real-time intracorporeal image in the patient's coordinate system and to compare them with the positions assigned to the imported data (second data modality). The control unit is then adapted to output only those of the imported data whose positions are located within the coordinates of the field of view. In particular, the surgeon can use an input to show or hide certain modalities, for example, histological data.

In a preferred further development, the control unit can be adapted to permanently store a real-time recorded data set of the assistance system in the data information system and to be able to recall it later. The term “recall” is to be comprehended within the meaning of the disclosure that the state of the assistance system stored with the data set, in particular a posture or position of the robot arm, is restored.

Preferably, this data set comprises at least one real-time, navigated posture or position of the robot, preferably its set of joint angles, and/or the at least one real-time, intracorporeal image and its location, and/or a real-time, navigated position of the instrument(s), preferably in the patient's coordinate system. If the data set only contains the posture or position of the robot and of the instrument and the real-time intracorporeal image and its location, it is a monomodal data set, since it only contains the at least one image in the first data modality with respect to the patient. When this data set is recalled, the stored posture or position of the robot and/or of the instrument(s) is reset, and/or the at least one real-time intracorporeal image is output/displayed again. In this way, so-called “waypoints” of the assistance system can be reproduced and approached at any time during the procedure.

According to a further development, the data set is supplemented by a selection of data recorded and imported with the second data modality before and/or during the procedure. In this case, the data set is multimodal, as it contains data on the patient in both the first and second data modalities. The selection may include all imported data of the second data modality whose positions are within the field of view, or it may contain only a subset thereof.

A specific data set or “waypoint” can be recalled, either by approaching the navigated posture or position of the robot stored with this data set, thereby triggering the output of the remaining data of the data set by reaching this posture or position, or by selecting and/or activating the data set, thereby triggering the approaching of the posture or position of the robot and the outputting of the remaining data of the data set.

In order to be able to define the selection or subset mentioned above and/or to avoid overloading the aforementioned view with too much displayed data, a user interface of the assistance system has been adapted to optionally select or deselect elements of the data set. When selected, the elements are output with and/or assigned to the data set to be stored; when deselected, they are not output and/or assigned to the data set to be stored.

Preferably, the control unit is adapted to set a focus point of the visualization system on the patient and to control the robot arm in such a way that the position of the focus point is maintained in any orientation of the visualization system.

In order to improve clarity of the view and facilitate interpretation of the data the control unit is adapted, according to a further embodiment, to at least output or display the real-time, intracorporeal image and/or the data collected in the second data modality in a hierarchical order depending on their data modality and/or the size of a field of view and/or a viewing angle of a field of view and/or depending on a recording time.

According to a preferred further development, the control unit, in particular the data information system, is adapted to store the data sets in their chronological order and to recall them again, thereby making it possible to create a history of the procedure and to specifically output interim results of the procedure, including the data relevant to surgery.

According to a preferred further development, the control unit, in particular the data information system, is adapted to determine or record a delta (difference) between stored data sets, in particular to perform a measurement. The measurement can be performed in particular between images and/or imported data of the same data modality and the same location or position, but of different record times, in order to determine progress during the procedure. In particular, distances between the data sets or differences in the record time can be calculated automatically from the quantity of stored data sets. This information can be useful for the surgeon to perform measurements on the patient, even if the patient's anatomy has changed during surgery.

According to a preferred further development the control unit is adapted to mark an area and/or a coordinate within the real-time intracorporeal image and/or in the data recorded in the second data modality and different from the visualization system, assign a function and/or a property and/or a parameter to the marked area and/or the marked coordinate in the sense of a functional mapping, and store the marking and assignment in the data information system. For example, results of histological examinations on a CT scan of the patient optionally can be marked as tumor tissue or healthy tissue and displayed in different colors. The user can thus perform a real-time interpretation of functional data and display it directly and intuitively on the patient's anatomy.

According to a preferred further development, the control unit is adapted to reproduce operating settings on a digital twin of the assistance system depending on the at least one stored data set. Since the posture or position of the robot arm and the visualization system are stored in the respective data set, this information can be used to reproduce a specific recording device both in the real world and in the digital and, in this respect, a virtual twin.

The tasks of the present disclosure are solved with regard to a method for data visualization for a surgical assistance system according to the present disclosure in that the method comprises the steps of:

Navigating a visualization system that is connected to a robot arm, in particular at an terminal side of the robot arm, in particular mounted, with spatial reference to the patient, by means of a control unit; creating and providing, in particular digitally, at least one real-time, intracorporeal image of the patient by means of the visualization system; determining a location, in particular a position and an orientation, of the visualization system, and thus a location of the at least one real-time, intracorporeal image with spatial reference to the patient by means of a navigation system; assigning a location in a coordinate system of the patient to the real-time intracorporeal image, and storing the location and image by way of the control unit; importing data that has been recorded using a second data modality different from the visualization system into a data information system by means of a data interface; assigning a position in the coordinate system of the patient to the imported data; storing the position and the imported data in the data information system by means of the control unit; generating a view of the real-time intracorporeal image together with those of the imported data which, due to their position, are spatially arranged within the real-time intracorporeal image, via the control unit; and outputting the view via a display device, preferably only one, in particular a surgery monitor.

With regard to a computer-readable storage medium or a computer program, the tasks are solved by the storage medium and computer program, respectively, comprising instructions which, when executed by the computer, cause it to perform the steps of the method according to the present disclosure.

Any disclosure relating to the surgical assistance system according to the present disclosure applies to the method according to the present disclosure, and vice versa.

### BRIEF DESCRIPTION OF THE DRAWINGS

The present disclosure is explained in more detail below with reference to preferred embodiments and with the aid of figures, wherein:

FIG. 1 shows a view of a surgical assistance system according to a first preferred embodiment;

FIG. 2 shows a multimodal data set of an assistance system according to a preferred embodiment;

FIG. 3 shows a method for data visualization of a multimodal data set according to a preferred embodiment;

FIG. 4 shows a multimodal data set according to a further preferred embodiment;

FIG. 5 shows a multimodal data set according to a further preferred embodiment;

FIG. 6 shows a multimodal data set according to a further preferred embodiment;

FIG. 7 shows a multimodal data set according to a further preferred embodiment; and

FIG. 8 shows a multimodal data set according to a further preferred embodiment.

The figures are schematic in nature and are intended to aid understanding of the present disclosure. Identical elements are provided with the same reference signs. Features of different embodiments may be interchanged.

### DETAILED DESCRIPTION

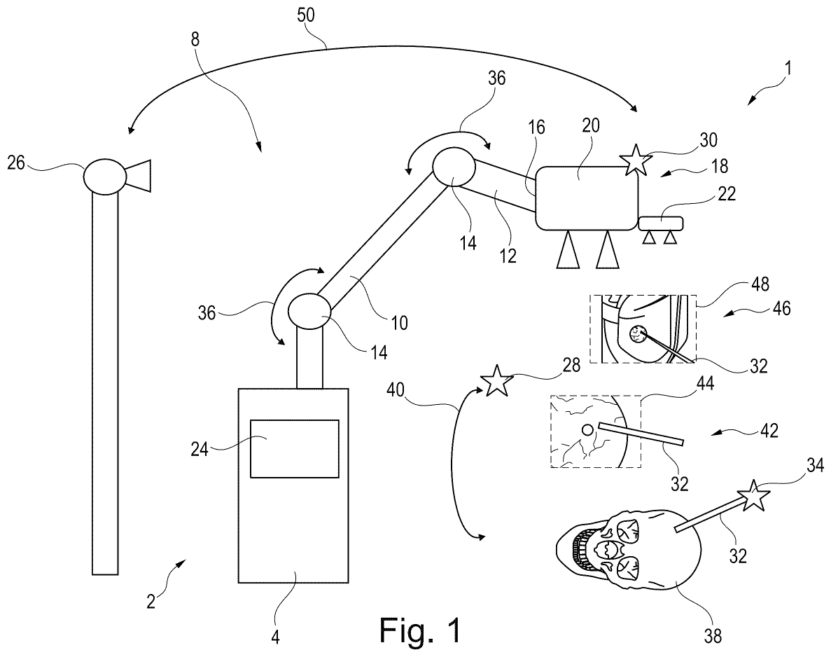

FIG. 1 shows a surgical assistance system 1 according to a preferred embodiment in a schematic side view. The assistance system 1 has a surgical robot 2 including a robot base 4, which is fixed locally in the embodiment shown. Alternatively, it can be executed to be mobile in order to utilize the robot 2 at different locations of a hospital operating room as required. In any case, the robot base 4 forms a local reference point to which a multi-segmented robot arm 8 that includes several robot arm segments 10, 12 is connected, which are each connected to one another via joints 14. In this way, the robot arm segments 10, 12 can be actively moved relative to one another and the robot arm 8 can be controlled as a whole.

A visualization system 18 of the assistance system 1 is attached to a terminal side 16 of the robot arm 8. In the embodiment shown, this comprises a surgical microscope 20 as well as an overview camera 22 as end effectors of the robot. In addition to the embodiment shown, at least one medical instrument may be provided as a rigid or movable end effector on the end segment 12 of the robot arm 8. In the embodiment shown, the assistance system 1 has a manually guided instrument 32 that is tracked by a navigation system of the assistance system 1.

The location (position and orientation) of the visualization system 18, and thus the respective location of the surgical microscope 20 and the overview camera 22, can be controlled and adjusted by the robot arm 8. A specially adapted control unit 24, which is provided on the robot base 4 and will be explained again below, serves as the central control unit for this purpose.

To move the robot arm 8 according to a user request, the assistance system 1 has an input device (not shown). This can be formed, for example, as a joystick or as a 3D space mouse that is adapted to receive both translational control command inputs for three perpendicular axes and rotational control command inputs around these three axes manually via haptic operation, and to forward them to the control unit 24 as computer-readable digital control signals and control commands, respectively, so that the control unit 24 can actively control the robot arm 8 in accordance with the control commands.

Furthermore, the assistance system 1 has a navigation system comprising several components. This includes a navigation camera 26, which in the embodiment shown is executed as an infrared stereo camera, a “tracker” 28, which is tracked by the navigation camera 26 and is firmly connected to the patient, and a “tracker” 30, which is firmly connected to the visualization system 18—and thus both firmly connected to the surgical microscope 20, the overview camera 22 and firmly connected to the end segment 12 of robot arm 8. The cameras 20, 22 are calibrated optically and their relative position to the tracker 30 is known and fixed, so that, if the location of the tracker 30 is known, the location of the cameras 20, 22 can also be determined via the navigation system. In addition, the navigation system includes a “tracker” 34 tracked by the navigation camera 26, which is firmly connected to the manually guided instrument 32. The navigation camera 26 is signal-connected to the control unit 24 and transmits the information on the tracked trackers 28, 30, and 34 to the control unit, which determines therefrom all necessary locations. Moreover, the navigation system includes angle sensors (not shown) on the joints 14, with which the joint angles 36/joint configuration of the multi-joint robot arm 8 are recorded in real time and transmitted to the control unit 24 for navigation.

The patient, who for reasons of clarity is represented in FIG. 1 solely by the tracker 28 attached to him, is registered with reference to his/her CT scan or MRI scan 38, a transformation 40 of the registration being stored in the control unit 24. The navigation system is thus set up with reference to a coordinate system of the patient so that the instrument 32 and the visualization system 18 and their cameras 20, 22 can be navigated with reference to the patient coordinate system.

During the procedure, the surgical microscope 20 and the overview camera 22 take real-time intracorporeal images 42, 46 with a respective field of view 44, 48 and make them available digitally to the control unit 24. The data modality of the two images 42, 46 is identical and, according to the disclosure, is referred to as the “first data modality”. The patient scan 38, on the other hand, is not real-time, but is recorded preoperatively. In addition, it has a data modality that differs from that of the visualization system 18, which is due to the fact that it is not optically captured data, as is the case for the images 42 and 46. Any data modality that differs from the first is referred to as the “second data modality” in accordance with the disclosure. One could also speak of an “additional/supplementary/further data modality.”

A multimodal data set of the assistance system 1 according to the present disclosure, which, alternatively, can be referred to as multimodal waypoint (“MMW”), according to FIG. 1, thus comprises the location 50 of the robot arm 8 determined from tracking tracker 30 or joint angles 36, and the locations of the surgical microscope 20 and the overview camera 22 determined therefrom. Moreover, it includes the location of the instrument 32 determined from tracking the tracker 34, as well as the location of the patient determined from tracking tracker 28. As real-time data of the “first data modality” it includes the real-time intracorporeal images 42 and 46, their location relative to the patient (tracker 28), and their fields of view 44, 48. The data set of the assistance system 1 comprises the patient scan 38 as preoperatively recorded data with a “second data modality” that differs from the visualization system 18.

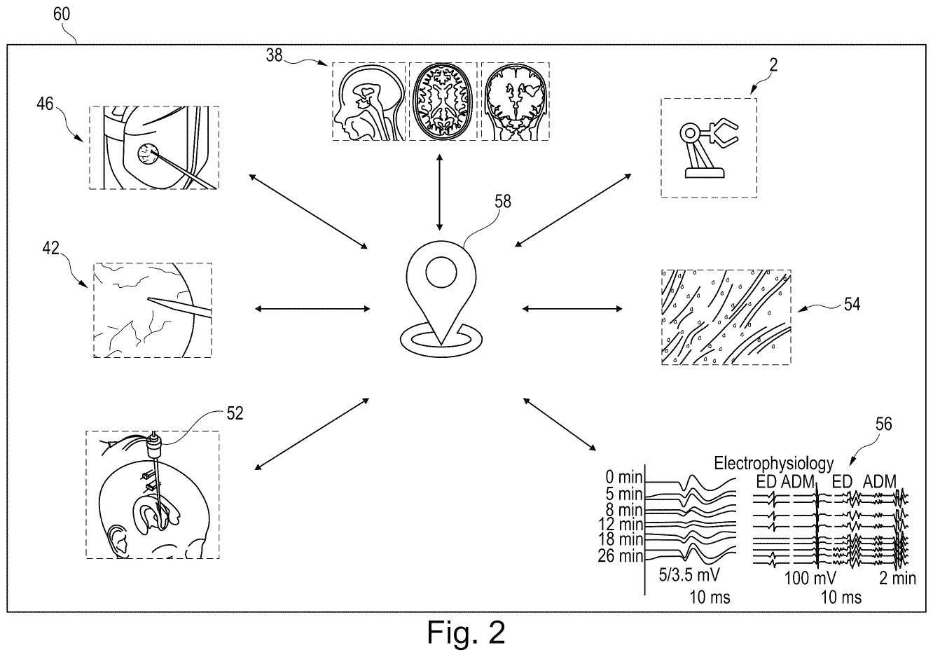

FIG. 2 shows a multimodal data set 60 of an assistance system according to a further preferred embodiment in compact form. A visualization system of the assistance system on which the data set according to FIG. 2 is based, is supplemented by a navigated endoscope camera 52 compared to the assistance system according to FIG. 1. The data set according to FIG. 2 therefore has additional real-time, intracorporeal images of the “first data modality” and a real-time image of the endoscope. In addition, the data set is supplemented by histological image data 54 and electrophysiological measurement data 56. Both represent external, non-real-time data with a “second data modality” that differs from the visualization system 18, which cannot be detected by the navigation system during their generation and initially therefore are not navigable.

As disclosed, these “second data modality” data, in this case the histological image data 54 and the electrophysiological measurement data 56, also are assigned a respective position in a coordinate system 58 of the patient, which is symbolized by the double arrows pointing to the coordinate system 58.

A multimodal data set—or MMW waypoint—thus consists, as disclosed, of a series of heterogeneous surgical data organized temporally and spatially according to a fixed reference system: the patient. In order to also make, in addition to images generated in real time and visualized live by the visualization system 18 (“first data modality”), data visible that are not generated in real time and generically is not part of the images and therefore cannot be navigated initially (“second data modality”), a specific location or coordinate in the patient's anatomy, i.e., in his/her coordinate system 58 is assigned, using a method for data visualization in accordance with the disclosure.

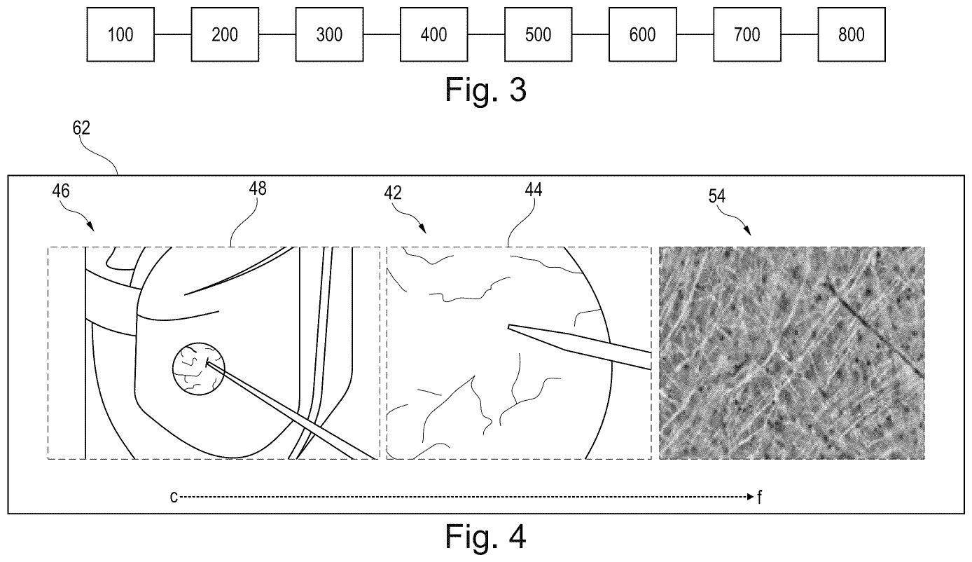

FIG. 3 shows such a method for data visualization according to a preferred embodiment. Based on the data set 60 according to FIG. 2, a complete sequence is shown, starting with step 100 “Navigating the visualization system 18” and ending with step 800 “Outputting a view of the data set 60.” Starting point is the registered patient.

In a first step 100, the visualization system 18 is navigated into the area of the procedure with spatial reference to the patient. This can be done, for example, through interaction of the aforementioned input device with the control unit. In a step 200, the intracorporeal overview-, surgical microscope- and endoscope-images are generated continuously and in real time via the visualization system 18 and are made available digitally to the control unit 24. In step 300, the location of the visualization system 18—and thus all locations of all real-time intracorporeal images—is determined by the navigation system with spatial reference to the patient. In step 400, the real-time intracorporeal images are assigned their respective locations in the patient's coordinate system 58 by the control unit and stored in a data information system of the control unit. In the further course of the process, it is to be assumed that data of a second data modality are available, for example, the digitally received result/image of a histological examination of a tissue sample previously taken from the patient. This is followed a priori by step 500, in which this data is imported into the data information system via a data interface. As already mentioned above, in step 600, this data is assigned a position in the patient's coordinate system 58 and stored in the data information system. This can be done, for example, by the surgeon assigning them a position in the real-time image—and thus on the patient—via a user interface, for example via a touchscreen or via a user interface on the input device or manually guided instrument 32. Alternatively, the data a priori can be placed in a predetermined manner in the center or at another predetermined position within the real-time image and subsequently moved by the surgeon. If, however, the data is recorded directly in the area of surgical procedure, for example, is electrophysiological measurement data at a surgical area of the brain, the surgeon can use his navigated instrument, for example, to specify the position at which the data is to be recorded. Via the record location the position of the data in the coordinate system 58 is then determined directly and stored in the data information system. In the next step 700, a view of the real-time intracorporeal image is generated jointly with that of the imported data, which are spatially arranged within the real-time intracorporeal image due to their previously assigned position. In step 800, the view is then output on a single display device, in particular a single surgery monitor.

A multimodal data set or waypoint generated and stored in this way can, of course, be recalled and explored by the surgeon at any time from the quantity of stored multimodal data sets. Recalling the data then causes all data stored in the multimodal data set to be visualized jointly on the patient's body, regardless of data modality. If the visualized data consist of stored images or is suitable for this purpose for another reason, it can be visualized hierarchically from coarse (c) to fine (f), or vice versa. This is illustrated in FIG. 4 for a data set 62, for which the image 46 from the overview camera 22 with a large field of view 48 is visualized on the left, the image 42 of the surgical microscope 20 with the significantly smaller field of view 44 is visualized in the middle, and the histological image 54 with the smallest field of view is visualized on the right.

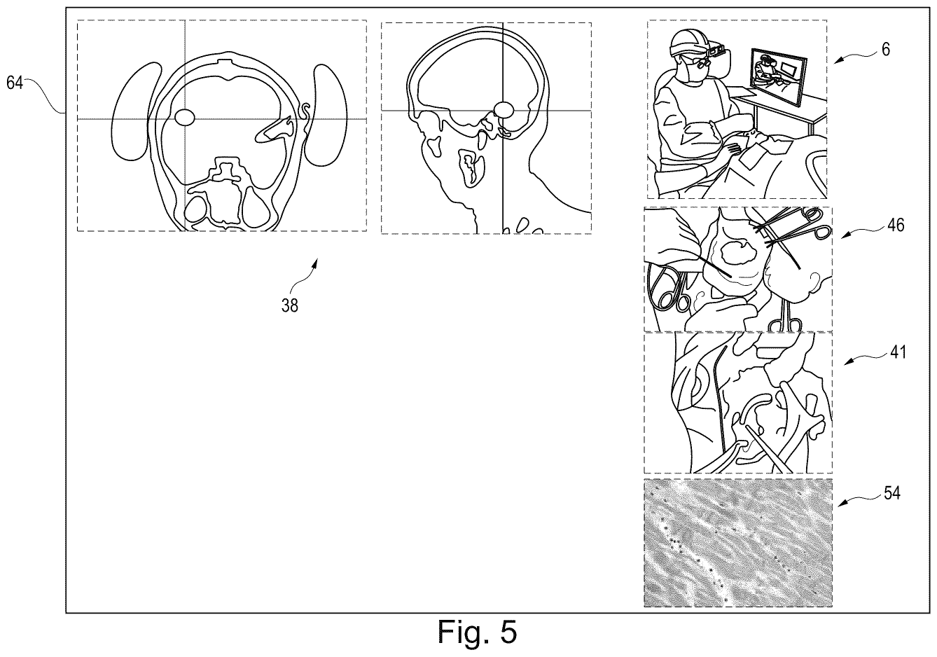

FIG. 5 illustrates another possibility offered by the multimodal data sets disclosed herein. Here, the aforementioned real-time images 42, 46 of the surgical microscope 20 and the overview camera 20, as well as a real-time, extracorporeal image 62 of the surgeon, the stored histological image 54, and the position of the data set or waypoint 64 are visualized within the context of patient scan 38. The surgeon can thus observe the position of the data set or waypoint 64 directly on the patient scan 38 and, if desired, recall a specific other data set or waypoint corresponding to its anatomical position by selecting a specific anatomical location on the patient scan 38. Alternatively, the surgeon may recall another data set or waypoint by setting or reproducing the recording conditions of a stored data set or waypoint. In this case, the robot 2 and the visualization system 18 are configured corresponding to the joint angle 36 stored in the recalled data set or waypoint or the stored location of the tracker 30. This makes it possible to reproduce visualization conditions exactly. This function can be particularly useful for comparing a specific anatomical position during the course of the surgery.

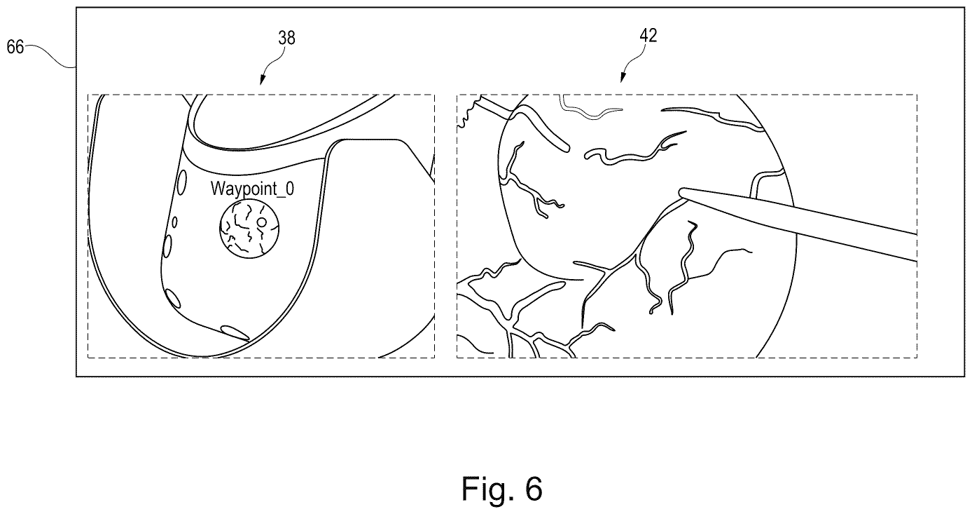

As already mentioned, the surgeon may recall any data set or waypoint and select, as required, which data from the data set is to be displayed. Selected data is displayed, whereas deselected data is not. Depending on the indication, the surgeon can thus freely choose between all available images and other imported data or functional data stored in the data set or waypoint. FIG. 6 shows an example of a multimodal data set 66 for which only the real-time image 42 of the surgical microscope 20, the instrument 32 positioned therein, and the preoperative patient scan 38, in which the position of the instrument 32 is visualized, have been selected for display/output.

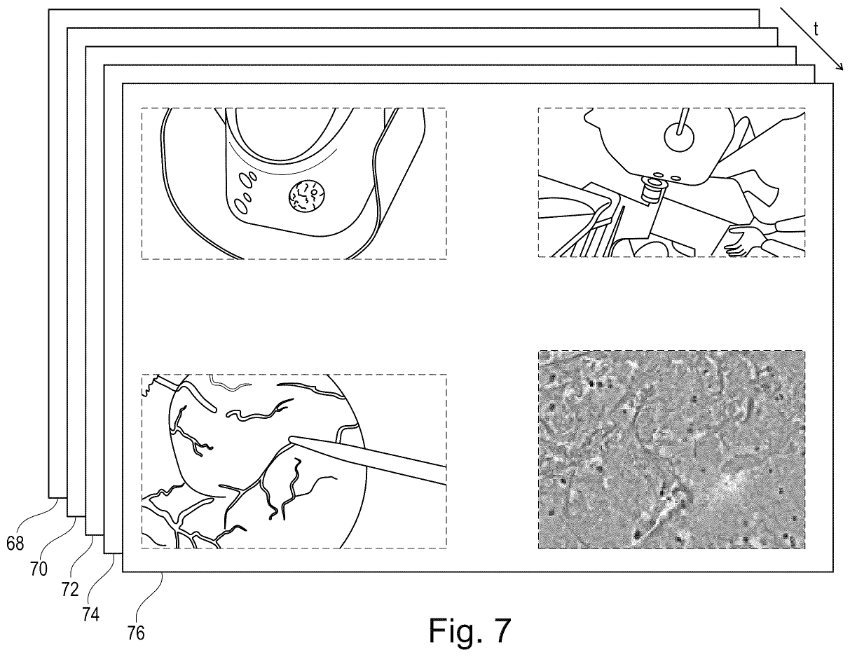

According to FIG. 7, a large number of multimodal data sets or waypoints 68, 70, 72, 74, 76 may arise and be stored during surgery. The data sets or waypoints 68, 70, 72, 74, 76 can be used to obtain a comprehensive overview on the procedure and to document it clearly. They are stored in the data information system according to their time of recording, i.e., according to FIG. 7 as a function of time t, and can be displayed according to a spatial hierarchy natively present in the data. This information is particularly useful for creating comprehensive and meaningful surgical documentation. In contrast to image data of conventional assistance systems, the data sets or waypoints 68, 70, 72, 74, 76 in accordance with the disclosure are organized both temporally and spatially. In addition, the data sets or waypoints 68, 70, 72, 74, 76 naturally can also be organized hierarchically internally, as already explained by way of FIG. 4.

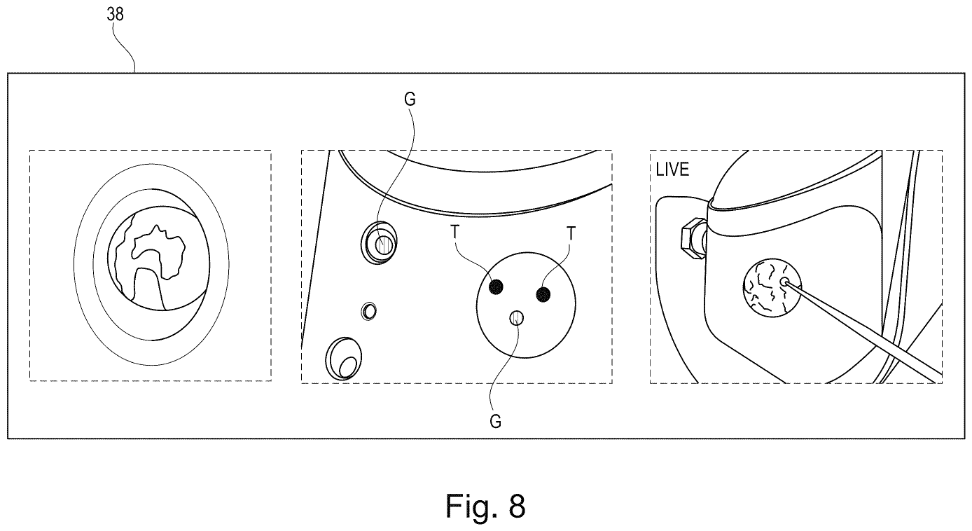

FIG. 8 shows the use of a multimodal data set or waypoint 78 for functional mapping. The surgeon can use the data set or waypoint 78 for live interpretation of functional data and display it intuitively on the patient's anatomy. As an example, FIG. 8 shows the results of a histological examination mapped directly onto the patient's anatomy. In reality, the mapping can be done using color marking, for example. An area marked with a T, for example, indicates tumorous tissue, while areas marked with a G indicate healthy tissue. The multimodal data set or waypoint 78 mapped in this functional manner contributes to optimal tumor resection.

### LIST OF REFERENCE SIGNS

1 Surgical assistance system
2 Robot
4 Robot base
8 Robot arm
10, 12 Robot arm segment
14 Joint
16 End section of robot arm
18 Visualization system
20 Surgical microscope
22 Overview camera
24 Control unit
26 Navigation camera
28 Patient tracker
30 Tracker of visualization system
32 Surgical instrument
34 Surgical instrument tracker
36 Patient scan
40 Transformation of registration
42 Image of surgical microscope
44 Field of view of surgical microscope
46 Image of overview camera
48 Field of view of overview camera
50 Robot arm location
52 Endoscope
54 Histological data
56 Electrophysiological data
58 Patient coordinate system
60 Multimodal data set
62 Image of camera of surgeon
64-78 Multimodal data set
100 Step of navigating visualization system
200 Step of creating and providing data of a first data modality
300 Step of determining the location in the visualization system
400 Step of assigning location
500 Step of importing data of a second data modality
600 Step of assigning position
700 Step of generating view
800 Step of outputting view

1.-15. (canceled)

16. A surgical assistance system for use in a surgical procedure on a patient, the surgical assistance system comprising:
a surgical robot;
a robot base as a local connection point of the surgical robot and a robot arm movably connected to the robot base and including at least one robot arm segment;
a visualization system connected to the robot arm and adapted to generate and provide at least one real-time intracorporeal image;
a navigation system adapted to determine, with spatial reference to the patient, at least one location of the visualization system and thus a location of the at least one real-time intracorporeal image; and
a control unit adapted to assign and store a location in a coordinate system of the patient to the at least one real-time intracorporeal image,
the control unit being configured to:
import data recorded in at least one second data modality different from the visualization system into a data information system of the control unit via a data interface,
assign at least a position to imported data in the coordinate system and store the imported data together with the position in the data information system,
generate a view of the at least one real-time intracorporeal image together with the imported data that are spatially arranged within the at least one real-time intracorporeal image, and output the view via a monitor.

17. The surgical assistance system according to claim 16, wherein the at least one second data modality comprises at least two second data modalities, and the imported data are recorded in the at least two second data modalities.

18. The surgical assistance system according to claim 16, wherein the data recorded with the at least one second data modality are image data and/or measurement data of the patient and/or operating data of the surgical robot.

19. The surgical assistance system according to claim 16, wherein:
the control unit is configured to permanently store a real-time-recorded data set of the surgical assistance system in the data information system,
the real-time-recorded data set comprises at least one real-time, navigated position of the surgical robot and the at least one real-time intracorporeal image and its location and the imported data, each in the coordinate system of the patient,
the control unit is configured to recall the real-time-recorded data set of the surgical assistance system from the data information system, and
when the real-time-recorded data set is recalled, a posture or position of the surgical robot is reset and the at least one real-time intracorporeal image and the imported data are output again.

20. The surgical assistance system according to claim 19, further comprising a user interface configured to selectively select elements of the data set for output or to deselect elements of the data set from the output.

21. The surgical assistance system according to claim 16, wherein the control unit is adapted to output at least the intracorporeal image of the visualization system and/or the data recorded in the second data modality in an ordered manner depending on their data modality, and/or depending on a size of a field of view and/or depending on a viewing angle of a field of view and/or depending on a recording time.

22. A method for data visualization during a surgical procedure on a patient using a surgical assistance system, the method comprising the steps of:
navigating a visualization system connected to a robot arm of a surgical robot with spatial reference to a patient by a control unit,
creating and providing at least one real-time intracorporeal image by the visualization system,
determining a location of the visualization system, and thus a location of the at least one real-time intracorporeal image, with spatial reference to the patient, by a navigation system, and
assigning a location in a coordinate system of the patient to the at least one real-time intracorporeal image and storing the location and image in a data information system of the control unit by means of the control unit,
importing data that is recorded with at least one second data modality different from the visualization system into the data information system by a data interface of the control unit,
assigning a position in the coordinate system to the imported data and storing the position and the imported data by the control unit,
generating a view of the at least one real-time intracorporeal image together with the imported data which, due to their position, are spatially arranged within the at least one real-time intracorporeal image, via the control unit, and
outputting the view via a monitor.

23. The method for data visualization according to claim 22, wherein:
the at least one second data modality comprises at least two second data modalities, and
the imported data are recorded in the at least two second data modalities.

24. The method for data visualization according to claim 22, further comprising the steps of:
permanently storing a real-time-recorded data set in the data information system by an operating interface and the control unit; and
recalling the real-time-recorded data set from the data information system by an operating interface and the control unit,
wherein:
the real-time-recorded data set contains at least one real-time navigated position of the surgical robot and the at least one real-time intracorporeal image and its location and the imported data, in each case in the coordinate system of the patient, and
wherein when real-time-recorded data set is recalled, a posture or position of the surgical robot is reset, and the at least one real-time intracorporeal image and the imported data are output again.

25. The method for data visualization according to claim 24, further comprising the step of recalling the real-time-recorded data set from the data information system,
wherein the step of recalling the real-time-recorded data set is triggered when the surgical assistance system moves to a position of the real-time-recorded data set via the control unit.

26. The method for data visualization according to claim 24, further comprising the step of assigning a selection of data recorded before the surgical procedure and/or during the surgical procedure with the second data modality to the stored data set by a user interface and the control unit.

27. The method for data visualization according to claim 24, further comprising the step of displaying a position of the stored data set in a scan or an image of the patient.

28. The method for data visualization according to claim 24, further comprising the step of creating a history of data sets.

29. The method for data visualization according to claim 24, further comprising the step of measuring differences between images and/or scans of a data modality and a location taken at different recording times.

30. The method for data visualization according to claim 24, further comprising the steps of:
marking an area and/or a coordinate within the at least one real-time intracorporeal image and/or in the data recorded in the second data modality different from the visualization system;
assigning a function and/or a property and/or a parameter; and
storing the marking and assignment in the data information system.

31. The method for data visualization according to claim 24, wherein the step of recalling the real-time-recorded data set is triggered when the robot arm moves to a position of said real-time-recorded data set.
